(19) United States
(12) Patent Application Publication
(10) Pub. No.: US 2017/0363620 A1
(43) Pub. Date: Dec. 21, 2017

(54) BIOMARKERS TO PREDICT NEW ONSET HEART FAILURE WITH PRESERVED EJECTION FRACTION (HFpEF)

(71) Applicant: Abbott Laboratories, Abbott Park, IL (US)

(72) Inventors: Agim Beshiri, Abbott Park, IL (US); Gillian Murtagh, Abbott Park, IL (US); Chris Watson, Dublin (IE); Eoin O'Connell, Dublin (IE); James O'Reilly, Dublin (IE); Stephanie James, Dublin (IE); Joe Gallagher, Dublin (IE); Mark Ledwidge, Dublin (IE); Ken McDonald, Dublin (IE)

(21) Appl. No.: 15/625,510

(22) Filed: Jun. 16, 2017

Related U.S. Application Data
(60) Provisional application No. 62/351,741, filed on Jun. 17, 2016.

(51) Int. Cl. G01N 33/53; C07K 16/40; G01N 33/577

(52) U.S. Cl. CPC G01N 33/53; G01N 33/577; C07K 16/40; G01N 2800/325

(57) ABSTRACT

The invention provides methods of detecting a natriuretic protein, a troponin, and galectin-3 in samples from subjects in order to determine risk of developing new onset heart failure with preserved ejection fraction (HFpEF).

### CROSS-REFERENCE TO RELATED APPLICATION(S)

This application claims priority to U.S. Provisional Patent Application No. 62/351,741, filed on Jun. 17, 2016, the entire contents of which are fully incorporated herein by reference.

### TECHNICAL FIELD

This disclosure relates to determining the risk of a subject developing new onset heart failure with preserved ejection fraction (HFpEF).

### BACKGROUND OF THE INVENTION

New onset heart failure with preserved ejection fraction (HFpEF) is characterized by progressive onset of cardiac remodeling and ventricular dysfunction, which provides opportunities to detect these manifestations of the disease earlier, thereby enabling timely intervention. The ability to predict new onset HFpEF is an important component of disease prevention strategies.

There is a need for methods determining risk of developing new onset heart failure with preserved ejection fraction (HFpEF). The invention described herein provides such methods.

### BRIEF SUMMARY OF THE INVENTION

The invention provides method of detecting a natriuretic peptide, a troponin, and galectin-3 in a sample obtained from a subject, which method comprises: (a) obtaining a sample from the subject, (b) detecting whether a natriuretic peptide, a troponin, and galectin-3 are present in the sample by contacting the sample with (i) an antibody that specifically binds to the natriuretic peptide, (ii) an antibody that specifically binds to the troponin, and (iii) an antibody that specifically binds to galectin-3.

The invention also provides a method of determining the risk of a subject developing new onset heart failure with preserved ejection fraction (HFpEF), which method comprises: (a) obtaining a sample from a subject, (b) contacting the sample with (i) an antibody that specifically binds to a natriuretic peptide, (ii) an antibody that specifically binds to a troponin, and (iii) an antibody that specifically binds to galectin-3, (c) detecting binding of the antibodies of (i), (ii), and (iii) of step (b) to a natriuretic peptide, a troponin, and galectin-3, respectively, (d) quantifying the amounts of the natriuretic peptide, the troponin, and galectin-3 in the sample, wherein increased levels of the natriuretic peptide, the troponin, and galectin-3 in the sample as compared to normal levels of the natriuretic peptide, the troponin, and galectin-3 indicate an increased risk of the subject developing HFpEF.

In one aspect of the above method of determining risk of a subject developing new onset HFpEF, the method further comprises administering a HFpEF treatment regimen or a HFpEF monitoring regimen to the subject determined to have an increased risk of developing new onset HFpEF.

The invention also provides a method of treating or monitoring a subject at risk of having heart failure with preserved ejection fraction (HFpEF), which method comprises: (a) obtaining a sample from a subject, (b) contacting the sample with (i) an antibody that specifically binds to a natriuretic peptide, (ii) an antibody that specifically binds to a troponin, and (iii) an antibody that specifically binds to galectin-3, (c) detecting binding of the antibodies of (i), (ii), and (iii) of step (b) to a natriuretic peptide, a troponin, and galectin-3, respectively, (d) quantifying the amounts of the natriuretic peptide, the troponin, and galectin-3 in the sample, and (e) administering a HFpEF treatment regimen or a HFpEF monitoring regimen to the subject if the natriuretic peptide, the troponin, and galectin-3 levels are higher in the sample as compared to the natriuretic peptide, the troponin, and galectin-3 levels in a control.

In one aspect of the above methods, the natriuretic peptide is brain natriuretic peptide (BNP).

In another aspect of the above methods, the troponin is troponin I.

In yet another aspect of the above methods, binding of the troponin to the antibody that specifically binds thereto is detected with a high-sensitivity troponin assay (hsTroponin).

In a further aspect of the above methods, the subject is a human having one or more risk factors for cardiovascular disease selected from the group consisting of uncontrolled high blood pressure, tobacco use, uncontrolled high low-density lipoprotein (LDL), diabetes mellitus, obesity, and physical inactivity.

In another aspect of the above methods, the subject does not exhibit any symptoms of cardiovascular disease.

In still yet another aspect of the above methods, an increased risk of the subject developing HFpEF is indicated when (i) the level of natriuretic peptide in the subject is at least 2-fold greater than the normal level of natriuretic peptide, (ii) the level of troponin in the subject is at least 2-fold greater than the normal level of troponin, and (iii) the level of galectin-3 in the subject is at least 2-fold greater than the normal level of galectin-3.

In a further aspect of the above methods, the sample is blood. In another aspect, the sample is plasma.

### DETAILED DESCRIPTION OF THE INVENTION

Embodiments of the present disclosure relate to methods for detecting a natriuretic peptide, a troponin, and galectin-3 in samples obtained from a subject, particularly for determining the risk of a subject developing new onset heart failure with preserved ejection fraction (HFpEF).

### 1. Definitions

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting.

The term “control subject,” as used herein, means a healthy subject, i.e. a subject having no clinical signs or symptoms of HFpEF. The control subject is clinically evaluated for otherwise undetected signs or symptoms of HFpEF, which evaluation may include routine physical examination and/or laboratory testing. A “control group” as used herein refers to a group of control subjects or healthy subjects, i.e. a group of subjects who have no clinical signs or symptoms of HFpEF.

The term “ejection fraction,” as used herein, refers to the fraction of outbound blood pumped from the heart with each heartbeat. It is commonly measured by echocardiogram and serves as a general measure of a subject's cardiac function. Left ventricular ejection fraction (LVEF) is the measurement of how much blood is being pumped out of the left ventricle of the heart (the main pumping chamber) with each contraction. Right ventricular ejection fraction (RVEF) is the measurement of how much blood is being pumped out of the right side of the heart to the lungs for oxygen. Typically, the term “ejection fraction” refers to left ventricular ejection fraction.

As used herein, the term “heart failure” refers to a condition in which the heart cannot pump blood efficiently to the rest of the body. Heart failure may be due to damage to the heart or narrowing of the arteries due to infarction, cardiomyopathy (primary or secondary), hypertension, coronary artery disease, valve disease, birth defects or infection. Heart failure can further be described as chronic, congestive, acute, decompensated, systolic or diastolic. The New York Heart Association (NYHA) classification describes the severity of the disease based on functional capacity of the patient; NYHA class can progress and/or regress based on treatment or lack of response to treatment. In heart failure, “increased severity” of cardiovascular disease refers to the worsening of disease as indicated by increased NYHA classification, to, for example, Class III or Class IV, and “reduced severity” of cardiovascular disease refers to an improvement of the disease as indicated by reduced NYHA classification, from, for example, class III or IV to class II or I.

Patients with an ejection fraction of 50 percent or higher are classified as having “heart failure with preserved ejection fraction” (HFpEF), whereas patients with an ejection fraction of less than 50 percent are classified as having “heart failure with reduced ejection fraction” (HFrEF) (see, e.g., Owan et al., N. Engl. J. Med., 355: 251-259 (2006); Hogg et al., J. Am. Coll. Cardiol., 43(3):317-2 (2004); Owan T, Prog. Cardiovasc. Dis., 47: 320-332 (2005); and Vasan R. S. et al., J. Am. Coll. Cardiol., 26: 1565-1574 (1995)).

The term “new onset,” as used herein, refers to the first appearance of the signs or symptoms of an illness.

“Galectin-3” or “Gal-3,” as used interchangeably herein, refers to a carbohydrate-binding lectin whose expression is associated with inflammatory cells including macrophages, neutrophils, and mast cells. Galectin-3 has been linked to cardiovascular physiological processes including myofibroblast proliferation, tissue repair, and cardiac remodeling in the setting of heart failure. Concentrations of galectin-3 have been used to predict adverse remodeling after a variety of cardiac insults (see, e.g., Felker et al., Circ. Heart Fail., 5(1): 72-78 (2012); Lok et al., Clin. Res. Cardiol., 99(5): 323-328 (2010); de Boer et al., Ann Med, 43(1): 60-68 (2011)).

“Natriuretic peptides (NPs),” as used herein, refers to hormones which are primarily secreted from heart and induce natriuresis (sodium excretion in the urine) and kaliuresis (potassium excretion in the urine). Four natriuretic peptides have been identified: atrial natriuretic peptide (ANP), B-type natriuretic peptide (BNP), C-type natriuretic peptide (CNP) and dendroaspis natriuretic peptide, a D-type natriuretic peptide (DNP). ANP is secreted by atria in the heart in response to high blood volume and is involved in homeostatic control of water, sodium, potassium, and adipose tissue. CNP is widely expressed in the vasculature, brain, bone and endothelium, but little if any CNP is present in the heart. CNP is a selective agonist for the B-type natriuretic receptor (NPRB), and dose not exhibit direct natriuretic activity. DNP has been reported to be present in human plasma and atrial myocardium and is elevated in plasma of human congestive heart failure.

“Brain natriuretic peptide” or “BNP,” as used interchangeably herein, refers to a neurohormone secreted mainly in the cardiac ventricles in response to volume expansion and pressure overload. Both ANP and BNP are activated in congestive heart failure, with BNP activated in patients with left ventricular (LV) dysfunction. BNP levels correlate with severity and prognosis of heart failure. BNP is synthesized by human cardiac myocytes as a 108-amino acid prohormone (proBNP), which is cleaved to the 32-residue BNP and the 76-residue N-terminal fragment of proBNP (NT-proBNP). Both BNP and proBNP are used as sensitive biomarkers of cardiac dysfunction. In acute coronary syndromes, increased concentrations BNP and proBNP are strong predictors of recurring myocardial infarction, heart failure, and death (see, e.g., Vuolteenaho et al., Adv. Clin. Chem., 40: 1-36 (2005); Maisel, A., Circulation, 105: 2328-2331 (2002); Tsutamoto et al., Circulation, 96: 509-516 (1997); Chen, H. H. and Burnett, J. C., Curr. Cardiol. Rep., 2: 198-205 (2000); Wallen et al., Heart, 77: 264-267 (1997); Yasue et al., Circulation, 90: 195-203 (1994); and U.S. Patent Application Publication 2013/0164767).

“Antibody” and “antibodies” refer to monoclonal antibodies, multispecific antibodies, bifunctional antibodies, human antibodies, humanized antibodies (fully or partially humanized), animal antibodies (such as, but not limited to, antibodies obtained or derived from a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (see, e.g., Wu et al., Nature Biotechnology, 25(11): 1290-1297 (2007), and International Patent Application Publication No. WO 2001/058956)), and functionally active epitope-binding fragments of any of the above. The term “bifunctional antibody,” as used herein, refers to an antibody that comprises a first arm having a specificity for one antigenic site and a second arm having a specificity for a different antigenic site, i.e., the bifunctional antibodies have a dual specificity.

The terms “antibody fragment” and “antibody fragments” refer to a portion of an intact antibody comprising the antigen-binding site or variable region. The portion does not include the constant heavy chain domains (i.e., CH2, CH3 or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein, the term “biomarker” refers to a measurable substance, the detection of which indicates a particular disease or risk of acquiring a particular disease. A “biomarker” may indicate a change in expression or state of the measurable substance that correlates with the prognosis of a disease. A “biomarker” may be a protein or peptide. A “biomarker” may be measured in a bodily fluid such as plasma. In the context of the method described herein, a “biomarker” can be a troponin (e.g., cardiac troponin) and/or copeptin.

As used herein, “diagnosis” and similar terms refer to the identification of a particular disease.

“Label” and “detectable label” mean a moiety attached, directly or indirectly, to an analyte-binding molecule (e.g., antibody or analyte-reactive fragment thereof) or an analyte to render the reaction between the analyte-binding molecule (e.g., antibody or analyte-reactive fragment thereof) and the analyte detectable, and the an analyte-binding molecule (e.g., antibody or analyte-reactive fragment thereof) or analyte so labeled is referred to as “detectably-labeled.” A label can produce a signal that is detectable, e.g., by visual or instrumental means. In this aspect, a label can be any signal-generating moiety, and sometimes is referred to herein as a reporter group. As used herein, the label (or signal-generating moiety) produces a measurable signal which is detectable by external means, e.g., by the measurement of electromagnetic radiation, and, depending on the system employed, the level of signal can vary to the extent the label is in the environment of the solid support, e.g., an electrode, microparticle or bead.

The term “low risk,” as used herein, is defined as less than or equal to a 10% chance, preferably less than a 5% chance, and more preferably less than a 2% chance of a subject developing new onset HFpEF. The term “moderate risk,” as used herein, is defined as greater than a 10% and less than a 30% chance of a subject developing new onset HFpEF. The term “high risk,” as used herein, is defined as greater than a 25% chance, preferably greater than or equal to a 30% chance, and more preferably greater than a 35% chance of a subject developing new onset HFpEF. It should be noted that the ranges and cutoff points recited herein in connection with the terms “low risk,” “moderate risk,” and “high risk” may vary depending upon the specific study utilized in order to gather the relevant data in connection with risk assessment. Further, it should be noted that these cutoff points relate to event risk and not relative risk.

“Predetermined cutoff,” “cutoff,” “predetermined level,” and “reference level” as used herein refer to an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels and reference levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “biological sample,” “test sample,” “specimen,” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing urine, blood, serum and plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

As used herein, the terms “prognosis,” “prognosticate,” and related terms refer to the description of the likely outcome of a particular condition, e.g., heart failure, HFpEF, and the like. For example, in a subject with suspected new onset HFpEF, measurement of plasma troponin I, galectin-3, and BNP concentrations enables determination of risk of short-term mortality, because plasma TnI, galectin-3, and BNP concentrations correlate with an increased risk of developing new onset HFpEF.

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). Preferably, the subject is a human.

The terms “treat,” “treated,” or “treating” as used herein refers to a therapeutic method wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

The term “troponin” refers to a complex of three regulatory proteins (i.e., troponin C, troponin I, and troponin T) that is located on the thin filament of the muscle contractile apparatus and plays an integral role in contraction of skeletal and cardiac muscle. Troponin I is a muscle protein which may be used in the determination of myocardial damage subsequent to or during, for example, a myocardial infarction. The other two subunits (i.e., T and C) also are immobilized on the thin myofilaments with troponin I in cardiac and skeletal muscle tissue. The cardiac forms of troponin C (cTnT) and troponin I (cTnI) are cardiac regulatory proteins that control the calcium-mediated interaction between actin and myosin. While expression of cTnT has not been identified outside of myocardium, cTnT is expressed to small extent in skeletal muscle (see, e.g., Bodor et al., Clin. Chem., 41:1710 (1995); and Ricchiuti et al., Clin. Chem., 44:1919 (1998)).

### 2. Detection of Natriuretic Peptide, Troponin, and Galectin-3

In one embodiment, the invention provides a method of detecting a natriuretic peptide, a troponin, and galectin-3 in a sample obtained from a subject. The method comprises (a) obtaining a sample from the subject, (b) detecting whether a natriuretic peptide, a troponin, and galectin-3 are present in the sample by contacting the sample with (i) an antibody that specifically binds to the natriuretic peptide, (ii) an antibody that specifically binds to the troponin, and (iii) an antibody that specifically binds to galectin-3.

Any suitable sample can be obtained from the patient. As defined herein, suitable samples include, for example, blood, serum, urine, saliva, lung tissue, pleural fluid, and cardiac tissue. Preferably, the sample is blood. More preferably, the sample is plasma. Plasma may be obtained by anti-coagulating blood with EDTA, sodium heparin, lithium heparin, sodium citrate, or sodium oxalate. Alternatively, the sample obtained from the patient is serum. In another embodiment, the sample can be whole blood.

The method described herein can detect any troponin expressed in humans, which includes troponin C (TnC), troponin I (TnI), troponin T (TnT), and cardiac forms thereof (i.e., cTnI and cTnT). In one embodiment, the method comprises detecting troponin I. Troponin I (TnI) is a 25 KDa inhibitory element of the troponin complex found in muscle tissue. TnI binds to actin in the absence of Ca²⁺, inhibiting the ATPase activity of actomyosin. The TnI isoform expressed in cardiac tissue (cTnI) is 40% divergent from skeletal muscle TnI, allowing both isoforms to be immunologically distinguished. The normal plasma concentration of cTnl is <0.1 ng/ml (4 pM). cTnl is released into the bloodstream following cardiac cell death; thus, the plasma cTnl concentration is elevated in patients with acute myocardial infarction (Benamer et al., Am. J. Cardiol., 82: 845-850 (1998)).

Troponin T has a molecular weight of about 37,000 Da. The troponin T isoform that is found in cardiac tissue (cTnT) is sufficiently divergent from skeletal muscle TnT to allow for the production of antibodies that distinguish both TnT isoforms. cTnT is considered a marker of acute myocardial damage (see, e.g., Katus et al., J. Mol. Cell. Cardiol., 21: 1349-1353 (1989); Hamm et al., N. Engl. J. Med., 327: 146-150 (1992); Ohman et al., N. Engl. J. Med., 335: 1333-1341 (1996); Christenson, Clin. Chem., 44: 494-501(1998); and U.S. Pat. No. 6,376,206).

Troponin C is the calcium-binding subunit of the troponin complex. Two distinct isoforms of the troponin C protein have been identified in vertebrate species: fast skeletal troponin C which is expressed exclusively in fast twitch skeletal muscle and slow/cardiac troponin C which is expressed in both cardiac and slow-twitch skeletal muscle (Schreier et al., J. Biol. Chem., 265(34): 21247-53 (1990). Both isoforms have a similar carboxyl-terminal domain. The binding of calcium to TnC abolishes the inhibitory action of TnI, thus allowing the interaction of actin with myosin, the hydrolysis of ATP, and the generation of tension.

As discussed above, galectin-3 (Gal-3) is a soluble 31 kDa β-galactoside-binding lectin. It is expressed in epithelial and inflammatory cells in several organs and it is located both intra- and extracellularly (see, e.g., Krześlak A. and Lipinska A., Cell Mol Biol Lett., 9:305-28 (2004); and Dumic et al., Biochim Biophys Acta., 1760: 616-35 (2006)). Gal-3 is involved in cellular functions related to cell adhesion, proliferation and differentiation (Dumic et al., supra). Gal-3 expression has been implicated in a wide range of physiological and pathological processes, including, but no limited to, cancer, fibrosis, renal function, and heart failure (see, e.g., Stoltze Gaborit et al., BMC Cardiovascular Disorders, 16: 117 (2016); Krześlak and Lipinska, supra, and Dumic et al., supra).

The method described herein also can detect any natriuretic peptide expressed in humans, including atrial natriuretic peptide (ANP), B-type natriuretic peptide (BNP), C-type natriuretic peptide (CNP) and dendroaspis natriuretic peptide, and D-type natriuretic peptide (DNP). In one embodiment, the method comprises detecting BNP. As discussed above, in humans, BNP is secreted by the heart through the coronary sinus, predominantly from the cardiac ventricles. The pre-pro peptide precursor of human BNP (hereinafter “human pre-proBNP”) is 134 amino acids in length and comprises a short signal peptide, which is enzymatically cleaved off to release the human pro peptide of BNP (hereinafter “human proBNP”) which is 108 amino acids in length. Human proBNP is further cleaved into an N-terminal pro peptide of human BNP (hereinafter “NT-proBNP”) which is 76 amino acids in length and the active hormone, human BNP (hereinafter “BNP,” “hBNP,” or “hBNP-32”), which is 32 amino acids in length. It has been suggested that each of NT pro-BNP, BNP, and proBNP can circulate in human plasma, and both NT pro-BNP and BNP and have been clinically evaluated for use in the management of congestive heart failure (See, Tateyama et al., Biochem. Biophys. Res. Commun., 185: 760-7 (1992); Hunt et al., Biochem. Biophys. Res. Commun., 214: 1175-83 (1995)).

A natriuretic peptide, troponin, and galectin-3 can be detected in the blood sample using any suitable method known in the art for detecting proteins in biological samples. Preferably, the methods described herein comprise detecting whether a natriuretic peptide, a troponin, and galectin-3 are present in the blood sample by contacting the blood sample with an antibody that specifically binds to the natriuretic peptide, an antibody that specifically binds to the troponin, and an antibody that specifically binds to galectin-3 or fragments (e.g., antigen-binding fragments) thereof. Antibodies which bind to troponins (including cardiac troponins), and components thereof, are known in the art (see, e.g., U.S. Pat. Nos. 8,030,026 and 8,835,120; U.S. Patent Application Publication 2007/0172888; and Krintus et al., Clin. Chem. Lab Med.; 52(11):1657-65 (2014)). Anti-troponin antibodies also are commercially available from sources such as, for example, Santa Cruz Biotech (Dallas, Tex,), Abbott Laboratories (Abbott Park, Ill.), Roche Diagnostics USA (Indianapolis, Ind.), and ThermoFisher Scientific, Inc. (Waltham, Mass.). Likewise, antibodies which bind galectin-3, and components thereof, are known in the art (see, e.g., U.S. Patent Application Publication 2010/0143954; Sumana et al., J. Clin. Diagn. Res., 9(11): EC07-11 (2015); and Gaze et al., Clin. Chem. Lab. Med., 52(6): 919-926 (2014)). Anti-Gal-3 antibodies also are commercially available from sources such as, for example, ThermoFisher Scientific, Inc. (Waltham, Mass.), Santa Cruz Biotech (Dallas, Tex.), Abbott Laboratories (Abbott Park, Ill.), and R & D Systems, Inc. (Minneapolis, Minn.). Antibodies which bind to ANP, and components thereof, are described in, e.g., Nagai, C. and Minamino, N., Anal. Biochem., 461: 10-16 (2014); and U.S. Pat. No. 5,156,977. Antibodies which bind to CNP, and components thereof, are described in, e.g., U.S. Pat. No. 7,919,255. Antibodies which bind to BNP, and components thereof, are disclosed in, e.g., U.S. Patent Application Publication 2013/0164767; Yandle, T. G. and Richards, A. M., Clin. Chim. Acta, 448: 195-205 (2015); and Collin-Chavagnac et al., Clin. Chem. Lab Med., 53(11): 1825-1837 (2015)). Antibodies that specifically bind to ANP, CNP, DNP, and BNP also are commercially available from sources such as, for example, ThermoFisher Scientific, Inc. (Waltham, Mass.), Santa Cruz Biotech (Dallas, Tex,), Abbott Laboratories (Abbott Park, Ill.), and R & D Systems, Inc., Minneapolis, Minn.).

Natriuretic peptides (e.g., BNP), troponin (e.g., troponin I), and galectin-3 can be detected in the blood sample using whole antibodies, as described herein, or antibody fragments, as described herein. In embodiments where a natriuretic peptide, a troponin, and galectin-3 are detected with antibody fragments, the fragment can be of any size so long as the fragment binds to a natriuretic peptide, a troponin, and galectin-3. In this respect, a fragment of an antibody that binds to a natriuretic peptide, a troponin, and/or galectin-3 comprises a heavy chain polypeptide and light chain polypeptide, each of which desirably comprises between about 5 and 18 (e.g., about 5, 6, 7, 8, 9, 10, 11, 12, 13, 14, 15, 16, 17, 18, or a range defined by any two of the foregoing values) amino acids.

Detecting binding of the anti-natriuretic peptide antibody (or antigen-binding fragment thereof), the anti-troponin antibody (or antigen-binding fragment thereof), and the anti-galectin-3 antibody (or fragment thereof) to the natriuretic peptide, the troponin, and galectin-3, respectively, can be performed using any suitable assay known in the art. Examples of suitable assays include, but are not limited to, immunoassay, such as sandwich immunoassay (e.g., monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, and the like.

The immunoassay methods of the present disclosure can be carried out in any of a wide variety of formats, descriptions of which are provided in, e.g., Asai, ed., Methods in Cell Biology Volume 37: Antibodies In Cell Biology, Academic Press, Inc. New York (1993), and Stites & Ten, eds., Basic and Clinical Immunology 7th Edition, (1991). A typical heterogeneous sandwich immunoassay employs a solid phase (as a solid support) to which is bound a first (capture) antibody reactive with at least one epitope on an analyte of interest that is an antigen. A second (detection) antibody is also reactive with at least one epitope on the analyte of interest that is an antigen. The second antibody may be conjugated to a detectable label that provides a signal that is measured after the detection antibody binds to the captured analyte. When a test sample containing the analyte contacts the first antibody, the first antibody captures the analyte of interest. The analyte of interest is contacted with the second antibody resulting in the formation of an immunodetection complex consisting of the first antibody, analyte of interest and second antibody, and the complex is bound to the solid phase. The signal generated by the second (detection) antibody is proportional to the concentration of the analyte of interest as determined by the rate of formation (k1) of the immunodetection complex versus the rate of dissociation of the immunodetection complex (k2). Heterophilic endogenous antibodies and any autoantibodies, which if present are unpredictable as to exactly where on the analyte of interest they will bind, can substantially interfere with binding of the first and/or second antibody, and thus with the resulting signal.

In one embodiment, the binding of a troponin to an anti-troponin antibody is detected with a high-sensitivity cardiac troponin assay (hs-Tn). By “high-sensitivity” is meant that the assay exhibits higher sensitivity for a cardiac troponin than other assays known in the art or commercially available. High-sensitivity troponin assays have been described in the art (see, e.g., Krintus et al., supra; and U.S. Pat. No. 8,835,120) and are commercially available from a variety of sources (e.g., ARCHITECT® high-sensitivity troponin I assay (Abbott Diagnostics, Lake Forest, Ill.; Elecsys troponin T high-sensitive assay (Roche Diagnostics GmbH, Mannheim, Germany); AccuTnI+3 troponin I assay (Beckman Coulter, Brea, Calif.), and assays available from SINGULEX®, Alameda, Calif.).

Assays used to detect a natriuretic peptide, a troponin, and galectin-3 in a sample obtained from a subject, such as a subject suspected of having HFpEF, can be adapted for use in a variety of automated and semi-automated systems (including those wherein the solid phase comprises a microparticle), such as those described in, e.g., in U.S. Pat. Nos. 5,089,424 and 5,006,309, and commercially marketed, e.g., by Abbott Laboratories (Abbott Park, Ill.) as ARCHITECT®.

Differences between an automated or semi-automated system as compared to a non-automated system (e.g., ELISA) include, for example, the substrate to which the first specific binding partner (e.g., an anti-analyte, monoclonal/polyclonal antibody (or a fragment thereof, a variant thereof, or a fragment of a variant thereof) or an anti-analyte DVD-Ig (or a fragment thereof, a variant thereof, or a fragment of a variant thereof) is attached, as well as the length and timing of the capture, detection and/or any optional wash steps. While a non-automated format, such as an ELISA, may require a relatively longer incubation time with sample and capture reagent (e.g., about two hours), an automated or semi-automated format (e.g., ARCHITECT®) may have a relatively shorter incubation time (e.g., approximately 18 minutes for ARCHITECT®). Similarly, while a non-automated format, such as an ELISA, may incubate a detection antibody, such as the conjugate reagent, for a relatively longer incubation time (e.g., about two hours), an automated or semi-automated format (e.g., ARCHITECT®) may have a relatively shorter incubation time (e.g., approximately four minutes for the ARCHITECT®).

Other platforms available from Abbott Laboratories that can be used in connection with the methods described herein include, but are not limited to, AxSYM®, IMx® (see, e.g., U.S. Pat. No. 5,294,404), PRISM®, EIA (bead), and Quantum™ II, as well as other platforms. Additionally, the methods described herein can be performed in other formats, for example, on electrochemical or other hand-held or point-of-care assay systems (e.g., i-STAT®, Abbott Laboratories) or an electrochemical immunoassay system that performs sandwich immunoassays. Immunosensors and their methods of manufacture and operation in single-use test devices are described in, for example, U.S. Pat. No. 5,063,081, and U.S. Patent Application Publication Nos. 2003/0170881, 2004/0018577, 2005/0054078, and 2006/0160164.

Other assay formats which may be used in connection with the method described herein include, for example, a rapid test, a Western blot, as well as the use of paramagnetic particles in, for example, an ARCHITECT® assay (see Frank Quinn, The Immunoassay Handbook, Second edition, edited by David Wild, pp. 363-367 (2001)), and other appropriate formats known to those of ordinary skill in the art.

The elements of the assays described above can also be used in the form of a kit. The kit may also comprise one or more containers (e.g., vials, bottles, or strips) comprising the assay components and reagents needed for performing the assay (e.g., washing, processing, and indicator reagents).

Methods other than immunoassay can be used to detect a natriuretic peptide, a troponin, and galectin-3 in accordance with the inventive method. In this regard, any method that can detect or quantify biomarkers in a sample can be used in the methods described herein. Such methods include physical and molecular biology methods in addition to immunological methods. For example, suitable physical methods include mass spectrometric methods, fluorescence resonance energy transfer (FRET) assays, chromatographic assays, and dye-detection assays. Suitable molecular biology methods include, but are not limited to, Northern or Southern blot hybridization, nucleic acid dot- or slot-blot hybridization, in situ hybridization, nucleic acid chip assays, PCR, reverse transcriptase PCR (RT-PCR), or real time PCR (e.g., taq-man PCR). Other methods to detect biomarkers include, e.g., nuclear magnetic resonance (NMR), fluorometry, colorimetry, radiometry, luminometry, or other spectrometric methods, plasmon-resonance (e.g. BIACORE), and one- or two-dimensional gel electrophoresis.

### 3. Determining Increased Risk of New Onset HFpEF

In another embodiment, the invention provides a method of determining risk of a subject developing new onset heart failure with preserved ejection fraction (HFpEF), which method comprises: (a) obtaining a sample from a subject, contacting the sample with (i) an antibody that specifically binds to a natriuretic peptide, (ii) an antibody that specifically binds to a troponin, and (iii) an antibody that specifically binds to galectin-3, (c) detecting binding of the antibodies of (i), (ii), and (iii) of step (b) to a natriuretic peptide, a troponin, and galectin-3, respectively, (d) quantifying the amounts of the natriuretic peptide, the troponin, and galectin-3 in the sample, wherein increased levels of the natriuretic peptide, the troponin, and galectin-3 in the sample as compared to normal levels of the natriuretic peptide, the troponin, and galectin-3 indicate an increased risk of the subject developing HFpEF.

Descriptions of the blood sample, antibodies that bind to a natriuretic peptide, a troponin, and Gal-3, methods for detecting antibody binding, and components thereof, set forth above in connection with the method of detecting a natriuretic peptide, a troponin, and Gal-3 also are applicable to the aforementioned method of determining risk of a subject developing new onset HFpEF.

In one embodiment, the subject is a human suspected of having HFpEF. A subject suspected of having HFpEF desirably has one or more risk factors for cardiovascular disease, including, but not limited to, uncontrolled high blood pressure, tobacco use, uncontrolled high low-density lipoprotein (LDL), diabetes mellitus, obesity, and physical inactivity. In another embodiment, the subject is a human that does not exhibit any symptoms of cardiovascular disease or heart failure.

Following detecting binding of a natriuretic peptide (e.g., BNP) to the anti-natriuretic peptide antibody, binding of a troponin (e.g., troponin I) to an anti-troponin antibody and binding of galectin-3 to an anti-Gal-3 antibody, the method comprises quantifying the amounts of a natriuretic peptide, a troponin, and Gal-3 in the sample. Any suitable method for quantifying antibody-antigen binding can be used in the methods described herein, a variety of which are known in the art. Typically, quantification of antibody-antigen binding is achieved by measuring a signal produced by a detectable label on the antibody or antigen, such as a radio- or fluorescence-label, and comparing the measured signal to either a standard curve for the protein(s) of interest (e.g., BNP, troponin I, and Gal-3) or by comparison to a reference standard for each protein. The reference standard may comprise anti-idiotypic antibodies, or a derivatized natriuretic peptide, troponin, and/or Gal-3 (e.g., derivatized with a polyethylene glycol).

A moiety on the label may not be detectable itself, but may become detectable upon reaction with yet another moiety (e.g., a secondary detectable label). For example, enzymes can be employed to produce a signal and/or to amplify a signal. As another example, the moiety can be a so-called quencher or an entity upon which a quencher acts. Use of the term “detectably-labeled” is intended to encompass these, and other means, of such labeling.

The detectable label can be any signal-producing substance known in the art, including, for example, an enzyme (e.g., horseradish peroxidase, alkaline phosphatase, alkaline peroxidase, glucose 6-phosphate dehydrogenase, and the like), a chromophore or chromogen (e.g., dyes that absorb light in the ultraviolet or visible region), a phosphor, a fluorescer, a fluorophor (e.g., fluorescent proteins such as green fluorescent protein, yellow fluorescent protein, red fluorescent protein, cyan fluorescent protein); a fluorescent label (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, quantum dots (e.g., zinc sulfide-capped cadmium selenide), a thermometric label, an immuno-polymerase chain reaction label; a phycobilin (e.g., phycoerythrin, R-Phycoerythrin, B-Phycoerythrin); biotin/avidin; a Xanthene derivative (e.g., fluorescein, rhodamine, Oregon green, eosin, Texas red); a cyanine derivative (e.g., cyanine, Cy dyes, indocarbocyanine, oxacarbocyanine, thiacarbocyanine, merocyanine); a naphthalene derivative (e.g., dansyl and prodan derivatives); a coumarin derivative; a oxadiazole derivative e.g., (pyridyloxazole, nitrobenzoxadiazole, benzoxadiazole); a Pyrene derivative (e.g., cascade blue); an oxazine derivative (e.g., Nile red, Nile blue, cresyl violet, oxazine 170); an acridine derivative (e.g., proflavin, acridine orange, acridine yellow); an arylmethine derivative (e.g., auramine, crystal violet, malachite green); a tetrapyrrole derivative (e.g., porphin, phtalocyanine, bilirubin)), a luminophore, a chemiluminescent compound (e.g., acridinium esters, thioesters, or sulfonamides; luminol, isoluminol, phenanthridinium esters, and the like), a radioactive compound (e.g., such as ³H, ¹²⁵I, ³⁵S, ¹⁴C, ³²P, and ³³P), and the like.

An acridinium compound can be used as a detectable label in a homogeneous chemiluminescent assay (see, e.g., Adamczyk et al., Bioorg. Med. Chem. Lett., 16: 1324-1328 (2006); Adamczyk et al., Bioorg. Med. Chem. Lett., 4: 2313-2317 (2004); Adamczyk et al., Biorg. Med. Chem. Lett., 14: 3917-3921 (2004); and Adamczyk et al., Org. Lett., 5: 3779-3782 (2003)). The acridinium compound can comprise at least one acridinium-9-carboxamide, at least one acridinium-9-carboxylate aryl ester, or any combination thereof. If the detectable label comprises at least one acridinium compound, the method also can comprise the use of a source of hydrogen peroxide, such as a buffer, solution, and/or at least one basic solution.

In one embodiment, the detectable label can be a phycobilin (e.g., phycoerythrin, R-Phycoerythrin, B-Phycoerythrin). R-Phycoerythrin, or PE, is useful as a fluorescence-based indicator for labeling analyte-binding molecules or other molecules in a variety of applications. R-Phycoerythrin absorbs strongly at about 566 nm with secondary peaks at 496 and 545 nm and emits strongly at 575 nm. R-Phycoerythrin is among the brightest known fluorescent dyes (see, e.g., Hayes, M. (ed.), Marine Bioactive Compounds: sources, Characterization and Applications, Springer (2012))

Detectable labels, labeling procedures, and detection of labels are described in detail in, for example, Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg.

In another embodiment, the method of determining risk of developing new onset HFpEF also involves comparing the levels of a natriuretic peptide, a troponin, and Gal-3 in a patient sample with a predetermined value. The predetermined value can take a variety of forms. For example, the predetermined value can be single cut-off value, such as a median or mean. The predetermined value can be established based upon comparative groups, such as where the risk of new onset HFpEF in one defined group is double the risk in another defined group. In yet another alternative, the predetermined value can be a range, for example, where the tested population is divided equally (or unequally) into groups, such as-a low-risk group, a medium-risk group, and a high-risk group, or into quadrants, the lowest quadrant being individuals with the lowest risk and the highest quadrant being individuals with the highest risk.

The predetermined value can depend upon the particular population selected. For example, an apparently healthy population will have a different normal range of biomarker expression levels than will a population comprised of patients with symptoms of cardiovascular disease or heart failure. In another embodiment, a population comprised of patients with heart failure with reduced ejection fraction (HFrEF) will have a different range of biomarker expression levels than will a population of HFpEF patients. Accordingly, the predetermined values selected may take into account the category in which an individual falls. Appropriate ranges and categories can be selected by those of ordinary skill in the art using routine methods. The level of a particular biomarker protein (e.g., cardiac troponin and copeptin) may be considered “elevated” if the antibody level measured is above a predetermined threshold level. In one embodiment, such a threshold level can be set to the 90th-percentile or to the 95th-percentile of a healthy control population. Preferably, the threshold level is established at the 95th-percentile of a healthy control population. In one embodiment, a risk of a subject developing new onset HFpEF is indicated when the level of each of the natriuretic peptide (e.g., BNP), the troponin (e.g., troponin I), and Gal-3 is at least 2-fold greater (e.g., 2, 5, 10, 15, 20, 25, 30, 35, 40, 45, 50-fold, or greater) than a predetermined normal level of the natriuretic peptide (e.g., BNP), the troponin (e.g., troponin I), and Gal-3.

In another embodiment, the method for determining risk of a subject developing HFpEF can be performed in conjunction with other methods for diagnosing HFpEF. It will be appreciated that several different criteria have been used to define and diagnose HFpEF, as diagnosing HFpEF has been challenging because it is largely involves excluding other potential noncardiac causes of symptoms suggestive of heart failure. Several different guidelines for the diagnosis of HFpEF have been described, all of which require the simultaneous and obligatory presence of signs and/or symptoms of HF, evidence of normal systolic LV function, and evidence of diastolic LV dysfunction or of surrogate markers of diastolic LV dysfunction such as LV hypertrophy, LA enlargement, atrial fibrillation, or elevated plasma NP levels (see, e.g., Paulus et al., Eur Heart J., 28: 2539-2550 (2007); Eur. Heart J., 19: 990-1003 (1998); Vasan, R. S. and Levy, D., Circulation, 101: 2118-2121 (2000); and Yturralde, R. F. and Gaasch, W. H., Prog Cardiovasc Dis, 47: 314-319 (2005)).

### 4. Methods of Treating or Monitoring HFpEF

In one embodiment, the present invention provides a method of treating or monitoring a subject at risk of having heart failure with preserved ejection fraction (HFpEF). The method comprises: (a) obtaining a sample from a subject, (b) contacting the sample with (i) an antibody that specifically binds to a natriuretic peptide, (ii) an antibody that specifically binds to a troponin, and (iii) an antibody that specifically binds to galectin-3, (c) detecting binding of the antibodies of (i), (ii), and (iii) of step (b) to a natriuretic peptide, a troponin, and galectin-3, respectively, (d) quantifying the amounts of the natriuretic peptide, the troponin, and galectin-3 in the sample, and (e) administering a HFpEF treatment regimen or a HFpEF monitoring regimen to the subject if the natriuretic peptide, the troponin, and galectin-3 levels are higher in the sample as compared to the natriuretic peptide, the troponin, and galectin-3 levels in a control.

a. Means for Monitoring the Subject Having HFpEF

The subject identified in the methods described above having levels of natriuretic peptide, the troponin, and galectin-3 levels higher in the sample than in a control can be closely monitored to determine response to therapy and to detect persistent or recurrent disease and metastasis. The subject may then be placed on a HFpEF monitoring regimen. Specifically, the subject may be administered a means for monitoring the effectiveness of any treatment regimens being used to treat the HFpEF as well as to assess the progress (or lack thereof) of the disease. HFpEF can be monitored using any number or variety of methods, including, but not limited to, diagnostic imaging techniques such as cardiac CT, MRI and echocardiography. Such methods can be combined with a physical examination and clinical history of the subject to enable the clinician to determine whether heart failure is progressing. The HFpEF monitoring regimen may involve conducting determining of natriuretic peptide, the troponin, and galectin-3 levels at periodic intervals (such periodic intervals being once a week, once a month, once every two months, once every three months, once every four months, once every five months, once every six months, once every seven months, once every eight months, once every nine months, once every ten months, once every eleven months, and once a year) or any combinations thereof.

b. Treatment Regiments for HFpEF

In another embodiment, the subject identified in the methods described above having levels of natriuretic peptide, the troponin, and galectin-3 levels higher in the sample than in a control are treated for HFpEF. Treatment of HFpEF is largely governed by management of associated conditions and symptoms. Any number or variety of treatment regimens can be used, including any treatment that reduces the risk of developing HFpEF. Risk for developing HFpEF is reduced when systolic and diastolic blood pressure is less than about 140/90 mm Hg, or less than about 130/80 mm Hg if the subject has diabetes or renal disease. Examples of suitable treatment regimens or therapeutics include, for example, mineralocorticoid receptor antagonists and diuretics (to treat volume overload). If diuretics are chosen, it will be appreciated that volume depletion should be avoided. Angiotensin receptor blockers (ARBs), angiotensin converting enzyme (ACE) inhibitors, calcium channel blockers, and beta blockers can be used to treat hypertension but may not alter clinical outcomes in HFpEF. In patients with diabetes mellitus who are at risk for heart failure, diuretic-based antihypertensive therapy, ACE inhibitors, beta-blockers, and glycemic control can be employed to prevent heart failure. Clinicians also can encourage lifestyle changes in patients at risk of developing HFpEF, such as, for example, weight loss, smoking cessation, regular exercise, and a healthy diet.

The following examples further illustrate the invention but, of course, should not be construed as in any way limiting its scope.

### EXAMPLE 1

This example demonstrates a method of detecting BNP, troponin I, and Gal-3 in samples obtained from a subject to determine risk of developing new onset HFpEF.

The utility of BNP, troponin I, and Gal-3 in predicting new onset HFpEF in asymptomatic, event free patients with cardiovascular disease (CVD) risk-factors was evaluated.

The study population consisted of 90 patients selected from within the longitudinal STOP-HF study (Ireland) which comprises asymptomatic patients with CVD risk factors. Thirty of these patients developed HFpEF over time, and were propensity matched 2:1 by age and sex to a cohort that did not develop HFpEF (n=60) over a similar time period. BNP, high sensitivity troponin I (hsTroponin I), and galectin-3 (Gal-3) were quantified in all patients at two time points. The median time between measurements was 1.2 years, and the median time between follow-up measurement and a future HFpEF event was 1.6 years.

Biomarker analysis of hsTroponin I and BNP at baseline and follow-up were statistically significant predictors of future new onset HFpEF, whereas galectin-3 at follow-up only was a significant predictor. A logistic regression model indicated that unadjusted biomarker combinations could significantly predict future HFpEF using both baseline (AUC 0.77 [0.68, 0.87]) and follow-up data (AUC 0.86 [0.79, 0.94]). Net reclassification improvement (NRI) between adjusted models indicated that it was not necessary to take account of patient medications at 80% sensitivity.

A simple clinical prediction rule to approximate the probability of future HFpEF development within the next 1-2 years was developed. Low and high risk was determined using BNP and galectin-3, and hsTroponin I was required to differentiate the intermediates. Applying this rule to the follow-up dataset yielded sensitivity and specificity values of 83% and 71%, respectively.

The results of this example confirms that detecting levels of a natriuretic peptide, a troponin, and Gal-3 in samples obtained from a subject can be used to determine risk of developing new onset HFpEF.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting a natriuretic peptide, a troponin, and galectin-3 in a sample obtained from a subject, which method comprises:
(a) obtaining a sample from the subject,
(b) detecting whether a natriuretic peptide, a troponin, and galectin-3 are present in the sample by contacting the sample with (i) an antibody that specifically binds to the natriuretic peptide, (ii) an antibody that specifically binds to the troponin, and (iii) an antibody that specifically binds to galectin-3.

2. A method of determining the risk of a subject developing new onset heart failure with preserved ejection fraction (HFpEF), which method comprises:
(a) obtaining a sample from a subject, (b) contacting the sample with (i) an antibody that specifically binds to a natriuretic peptide, (ii) an antibody that specifically binds to a troponin, and (iii) an antibody that specifically binds to galectin-3,
(c) detecting binding of the antibodies of (i), (ii), and (iii) of step (b) to a natriuretic peptide, a troponin, and galectin-3, respectively,
(d) quantifying the amounts of the natriuretic peptide, the troponin, and galectin-3 in the sample, wherein increased levels of the natriuretic peptide, the troponin, and galectin-3 in the sample as compared to normal levels of the natriuretic peptide, the troponin, and galectin-3 indicate an increased risk of the subject developing HFpEF.

3. The method of claim 2, further comprising administering a HFpEF treatment regimen or a HFpEF monitoring regimen to the subject determined to have an increased risk of developing new onset HFpEF.

4. A method of treating or monitoring a subject at risk of having heart failure with preserved ejection fraction (HFpEF), which method comprises:
(a) obtaining a sample from a subject,
(b) contacting the sample with (i) an antibody that specifically binds to a natriuretic peptide, (ii) an antibody that specifically binds to a troponin, and (iii) an antibody that specifically binds to galectin-3,
(c) detecting binding of the antibodies of (i), (ii), and (iii) of step (b) to a natriuretic peptide, a troponin, and galectin-3, respectively,
(d) quantifying the amounts of the natriuretic peptide, the troponin, and galectin-3 in the sample, and
(e) administering a HFpEF treatment regimen or a HFpEF monitoring regimen to the subject if the natriuretic peptide, the troponin, and galectin-3 levels are higher in the sample as compared to the natriuretic peptide, the troponin, and galectin-3 levels in a control.

5. The method of claim 3, wherein the HFpEF treatment regimen comprises mineralocorticoid receptor antagonists, diuretics, beta-blockers, calcium channel blockers, angiotensin receptor blockers (ARBs), angiotensin converting enzyme (ACE) inhibitors, lifestyle changes, or combinations thereof.

6. The method of claim 3, wherein the HFpEF monitoring regimen comprises a cardiac CT, magnetic resonance imaging (MRI), echocardiography, physical examination of the subject, clinical history of the subject, or combinations thereof.

7. The method of claim 1, wherein the natriuretic peptide is brain natriuretic peptide (BNP).

8. The method of claim 1, wherein the troponin is troponin I.

9. The method of claim 1, wherein binding of the troponin to the antibody that specifically binds thereto is detected with a high-sensitivity troponin assay (hsTroponin).

10. The method of claim 2, wherein the subject is a human having one or more risk factors for cardiovascular disease selected from the group consisting of uncontrolled high blood pressure, tobacco use, uncontrolled high low-density lipoprotein (LDL), diabetes mellitus, obesity, and physical inactivity.

11. The method of claim 10, wherein the subject does not exhibit any symptoms of cardiovascular disease.

12. The method of claim 2, wherein an increased risk of the subject developing HFpEF is indicated when (i) the level of natriuretic peptide in the subject is at least 2-fold greater than the normal level of natriuretic peptide, (ii) the level of troponin in the subject is at least 2-fold greater than the normal level of troponin, and (iii) the level of galectin-3 in the subject is at least 2-fold greater than the normal level of galectin-3.

13. The method of claim 1, wherein the sample is blood.

14. The method of claim 13, wherein the sample is plasma.
